(19) United States
(12) Patent Application Publication
(10) Pub. No.: US 2021/0060060 A1
(43) Pub. Date: Mar. 4, 2021

(54) COMPOSITIONS AND METHODS FOR THE TREATMENT OF LUNG EMPHYSEMA AND OTHER FORMS OF COPD

(71) Applicant: Emphysema Solutions BV, Beuningen (NL)

(72) Inventor: ROB JANSSEN, Beuningen (NL)

(21) Appl. No.: 16/961,147

(22) PCT Filed: Jan. 11, 2019
(86) PCT No.: PCT/NL2019/050016

§ 371 Date: Jul. 9, 2020

(30) Foreign Application Priority Data
Jan. 11, 2018 (NL) 1042709

(51) Int. Cl. A61K 33/34; A61K 31/727; A61K 9/00; A61P 11/00; A61K 31/506; A61K 31/203; A61K 33/06; A61K 31/122

(52) U.S. Cl. CPC A61K 33/34; A61K 31/727; A61K 9/007; A61K 31/122; A61K 31/506; A61K 31/203; A61K 33/06; A61P 11/00

(57) ABSTRACT

A composition for use in a method for the treatment of lung emphysema and other forms of COPD is provided comprising an active agent comprising a copper compound, preferably copper sulfate, and a glycosaminoglycan, preferably heparin, or a physiologically acceptable salt thereof. The composition is preferably administered via inhalation and/or via instillation.

### FIELD OF THE INVENTION

The present invention is in the field of pharmacotherapy. In particular, the invention relates to compositions and methods for the treatment of lung emphysema, with or without presence of airflow limitation, and other forms of chronic obstructive pulmonary disease (COPD).

### BACKGROUND OF THE INVENTION

COPD is one of the most prevalent non-communicable conditions. COPD is a complex clinical situation having as a common factor smoking-related, fixed airflow limitation, which does not change markedly over periods of several months of observation. Moreover, the airflow obstruction shows an abnormal rapid progressive deterioration with age. The disease course culminates in chronic respiratory symptoms.

COPD pathogenesis is characterized by chronic inflammation and accelerated loss of elastic fibers [1]¹. The chronic airflow limitation in COPD is caused by small airways disease, lung parenchymal destruction (i.e. emphysema) or a mixture of both [1]. Emphysema is the COPD phenotype characterized by excessive loss of elastin fibers in lung parenchyma due to protease/antiprotease and elastin degradation/repair imbalances. Although less appreciated than the role of elastin degradation in the pathogenesis of emphysema, accumulation of collagen in the lung parenchyma is another important pathogenic characteristic of emphysema [2]. ¹ Numbers in square brackets refer to the numbers in the reference list at the end of the description

Elastin, the main component of elastic fibers, is a unique protein that provides elasticity, resilience and deformability to the lungs, and it is therefore a basic requirement for breathing [3]. Elastin is mainly produced in utero and early childhood [4].

The production of elastin fibers starts with the synthesis of the elastin precursor tropo-elastin by several cell types [4]. Tropo-elastin is subsequently secreted in the extracellular matrix, transported to a fibril scaffold, aligned with many other tropo-elastin-proteins into polymers, and finally crosslinked with other tropo-elastin-polymers into mature and durable elastin fibers that are required to last a lifetime [4]. The crosslinking process is facilitated by the enzymes LOX and LOX like proteins (LOXL) 1 to 4 [4]. Fibulins 4 and 5 also play important roles in the development and maintenance of elastin fibers. Whereas prototype LOX and fibulin-4 are mainly involved in the initial development of elastin fibers, LOXL1 and fibulin-5 are essential to elastin repair.

The elastic properties of lungs are compromised by elastin degradation [4], which is enhanced in patients with COPD due to an imbalance between the protective effects of anti-proteases and the destructive properties of proteases [3]. Another driver of elastin degradation is imbalance between elastin degradation and elastin repair, given that damaged elastin fibers are more susceptible to further destruction by proteases than native fibers [5]. Furthermore, elastin fibers that are crosslinked by LOX enzymes are relatively resistant to proteases, whereas un-crosslinked proteins are readily degraded [6-8]. Accelerated pulmonary elastin degradation is an important pathogenic mechanism in emphysema leading to lung function loss [9].

Besides accelerated elastin loss, there is another problem in the extracellular matrix of patients with emphysema. It has been demonstrated that collagen levels in the lungs of patients with emphysema are elevated compared to controls [10], and inversely correlated to forced expiratory volume in one second (FEV₁) [11].

Copper serves as a cofactor in the activation of LOX enzymes (i.e. prototype LOX and LOXL1-4) [12]. Induced copper deficiency in chicks disrupts elastin crosslinking due to reduced LOX activity and leads to a net decrease in elastin content [12]. The reason for the lower elastin content in copper deficiency seems to be caused by enhanced degradation, since un-crosslinked tropo-elastin is much more susceptible to proteases than properly crosslinked elastin [12]. Copper repletion in copper-deficient chicks restores deposition of protease-resistant elastin fibers to near normal values [7].

Loss of elastin fibers causes COPD in the lungs, whereas it causes formation of wrinkles in the skin [13]. Copper-containing cream in healthy controls induces an increase of elastin crosslinks in the skin [14].

There is evidence suggesting copper shortage in lung emphysema. Emphysem-atous changes can be induced in rats and hamsters by feeding them a copper-deficient diet [15,16]. Copper deficiency caused 17% reduction of elastin content and 35% larger alveolar spaces in rats' lungs [15]. Copper repletion restored the ultrastructure of pulmonary elastin to near normal [15].

Expression of the pro-inflammatory cytokine tumor necrosis factor alpha (TNF-α) is enhanced in emphysema [17, 18]. Transgenic mice with lung-specific TNF-α over-expression develop emphysematous lesions [19]. It was concluded that copper deficiency occurs following chronic TNF-α induced lung inflammation and this likely plays an essential role in the inflammation-induced lung damage.

There is also a human study suggesting local copper deficiency in emphysem-atous areas. The protein copper metabolism domain containing-1 (COMMD1) is a key-regulator of copper metabolism [20]. It has been demonstrated that the levels of COMMD1 as well as active LOX, LOXL1 and LOXL2 are reduced in emphysema lungs [21].

Copper concentrations in exhaled breath condensate of patients with COPD are decreased and inversely related to FEV₁ [22]. This may suggest the presence of copper deficiency in COPD lungs. In line with this, individuals with Menkes disease, a genetic disorder of copper transport, may develop severe emphysema [23].

Although the prior art may suggest that copper is a useful stimulator of elastin repair and development in emphysematous lungs, there is one crucial problem that precludes the use of copper as a therapy in patients with emphysema. LOX enzymes are not only stimulators of elastin crosslinking but also of collagen crosslinking. Increased collagen crosslinking will lead to enhanced organization, maturation and thereby accumulation of collagen in emphysematous lungs, which is highly undesirable given the fact that collagen levels are already increased in patients with emphysema and will provoke a transition of lung emphysema to lung fibrosis, which is another devastating lung disease. Therefore, copper-induced stimulation of collagen accumulation teaches away from the use of copper as a therapy in patients with emphysema.

The most important complaints of patients with COPD are dyspnea on exertion and in later stages also at rest, and exercise intolerance. From a mechanistic point of view, it seems to be more appropriate to regard COPD as a syndrome rather than as a uniform disease entity. Airflow obstruction in COPD patients is caused by small airways disease, lung emphysema or a combination of both. Significant emphysema is also frequently present on computed tomography (CT) in (former) smokers with no COPD (i.e. absence of airflow obstruction).

It is clinically difficult to distinguish emphysema from chronic bronchitis because of similar symptoms of shortness of breath, cough and wheezing. In a substantial portion of patients, combinations of the characteristics ascribed to either chronic bronchitis or emphysema are present.

The Fleischner Society for Thoracic Imaging issued a statement describing CT-definable subtypes of COPD. The main pathologic categories that can be distinguished are airway wall thickening, bronchiectasis, small airways disease and emphysema.

It should be realized that these radiologic abnormalities can also be identified in individuals without COPD. Emphysema is characterized by irreversible lung damage. As a result, elasticity of the lung tissue is lost, causing airways to collapse and obstruction of airflow to occur. Chronic bronchitis is an inflammatory disease that begins in the smaller airways within the lungs gradually advances to larger airways. It increases mucus in the airways and increases bacterial infections in the bronchial tubes, which, in turn, impedes airflow.

Current pharmacological COPD therapy is able to ameliorate respiratory symptoms and the frequency of exacerbations, as well as to improve quality of life and exercise capacity [1]. Decelerating effects of inhalation therapy with long-acting broncho-dilators and corticosteroids on the rate of lung function decline have also been reported [2-4]. Unfortunately, inhaled bronchodilators and corticosteroids mainly target the airway component of COPD, and do not have as much favorable effects in emphysema-dominant as in airways-dominant COPD patients. However, the presence of emphysema on CT is an important finding, as it is strongly associated with mortality.

No single COPD intervention except for lung transplantation has been proven effective in recovering lung function [1]. Hence there is an urgent need to establish specific pharmacological therapy for the large group of individuals with emphysema.

WO 03/068187 A1 discloses the use of glycosaminoglycans, e.g. heparin, for the treatment of respiratory disorders such as COPD, in particular chronic airflow limitation (CAL).

WO 2012/073025 A1 discloses glycosaminoglycans such as heparin for use in the treatment and/or prevention of COPD, wherein, after administration to a subject, the heparin reduces inflammation in the lungs of the subject.

### SUMMARY OF THE INVENTION

The present invention is based on the unexpected finding that the combination of copper and certain glycosaminoglycans, in particular heparin, can be used to treat lung emphysema and other forms of COPD. The combination has a beneficial effect on the repair and development of elastin fibers in lungs of patients with emphysema and at the same time prevents copper-induced stimulation of collagen crosslinking.

Although the prior art discloses the use of heparin as an inhalation monotherapy to patients with COPD [24,25], it does not teach or suggest the synergistic value of adding heparin to copper inhalation therapy to further stimulate elastin repair/developmental processes through stimulation of tropo-elastin crosslinking and, more importantly, to prevent copper-induced stimulation of collagen crosslinking.

Accordingly, the present invention provides in one aspect a composition for use in a method for the treatment of lung emphysema and other forms of COPD comprising an active agent comprising a copper compound, and a glycosaminoglycan or a physiologically acceptable salt thereof. The use of administration by inhalation is particularly preferred.

In a preferred embodiment, the composition according to the invention is used as an additive to standard pharmacological COPD treatment which includes bronchodilators and immune-modulators, such as inhaled corticosteroids and oral macrolides.

In another aspect of the invention, a method of treatment of a subject suffering from lung emphysema or another form of COPD is provided which comprises administering to said subject a therapeutically active amount of a composition an active agent comprising a copper compound, and a glycosaminoglycan or a physiologically acceptable salt thereof.

The significance of the active ingredients of the composition according to the invention with respect to the repair and development of elastin fibers and the prevention of collagen accumulation will be more fully outlined in the detailed description which follows.

### BRIEF DESCRIPTION OF THE DRAWINGS

FIG. 1: Explanted right lung of a 55-year-old male patient with combined pulmonary fibrosis and emphysema. A. Gross anatomy of resection specimen, showing extensive changes throughout the lung, with advanced emphysema in the upper and middle lobes, bulla formation in the upper lobe, and micronodular pleural changes of the middle (*) and lower lobes, corresponding with extensive parenchymal fibrosis. B. Representative microscopy (Hematoxylin-eosin stain, 2.5× magnitude) of the upper lobe, displaying extensive emphysematous change (*) and mild, bland interstitial fibrosis. C. Representative microscopy (Hematoxylin-eosin stain, 10× magnitude) of the lower lobe, characterized by advanced fibrosis with architectural distortion and numerous fibroblastic foci (**), amounting to a usual interstitial pneumonia pattern. The middle lobe demonstrated a combination of emphysema and advanced fibrosis (not shown).

FIG. 2: Relative concentrations of elastin, collagen, (iso)desmosine (DES) and hydroxyproline (Hypro) in lungs of control subjects, of patients with emphysema, idiopathic pulmonary fibrosis and combined pulmonary fibrosis (CPFE; in basal and apical lung zones). Levels in control subjects are set at 100%.

FIG. 3: Relative copper concentrations in serum and exhaled breath condensate (EBC) in control subjects (set at 100%) and patients with emphysema and idiopathic pulmonary fibrosis (IPF).

FIG. 4: Relative copper concentrations in lung parenchyma of control subjects (set at 100%) and patients with emphysema, idiopathic pulmonary fibrosis (IPF) and combined pulmonary fibrosis and emphysema (CPFE; apical and basal lung regions).

FIG. 5: (Iso)desmosine (DES) levels in fibroblast medium (in vitro cell cultures) without additional copper (baseline) or (baseline copper concentration) +0.5, 1, 2, 4, 8, 16 and 32*baseline copper.

FIG. 6: Relative gene expression of lysyl oxidase (LOX), lysyl oxidase like 1 (LOXL1), elastin (ELN), fibulin-5 and levels of tropo-elastin, insoluble elastin, (iso)desmosine (DES) and collagen in fibroblasts (in vitro cell cultures) grown in baseline+8*baseline copper concentration alone, copper plus retinoic acid (RA), copper plus minoxidil and copper plus heparin.

FIG. 7: Relative levels insoluble elastin and (iso)desmosine (DES) in fibroblasts (in vitro cell cultures) grown in baseline+8*baseline copper concentration alone, copper plus vitamin K1, copper plus vitamin K2 and copper plus magnesium sulfate.

FIG. 8: Total lung capacity (TLC) and mean linear intercept (Lm) in control mice, copper mice and copper/heparin mice.

FIG. 9: (Iso)desmosine (DES), collagen and hydroxyproline (Hypro) in control mice, copper mice and copper/heparin mice.

FIG. 10: Microscopy (10× magnitude) of the lung from a mouse of the placebo group displaying extensive emphysematous changes.

FIG. 11: Microscopy (10× magnitude) of the lung from a mouse of the copper/heparin group displaying normal alveoli with no emphysematous changes.

FIG. 12: First measurement of particle size distribution of a 5 mL sodium chloride 0.9% solution with 5,000 IU heparin and 0.5 mg copper using laser diffraction analysis.

FIG. 13: Duplicate measurement of particle size distribution of a 5 mL sodium chloride 0.9% solution with 5,000 IU heparin and 0.5 mg copper using laser diffraction analysis.

FIG. 14: First measurement of particle size distribution of a 5 mL sodium chloride 0.9% solution with 100,000 IU heparin and 1.0 mg copper using laser diffraction analysis.

FIG. 15: Duplicate measurement of particle size distribution of a 5 mL sodium chloride 0.9% solution with 100,000 IU heparin and 1.0 mg copper using laser diffraction analysis.

### DETAILED DESCRIPTION OF THE INVENTION

Reactivation of pulmonary elastin fiber production and repair of damaged elastin fibers are prerequisites to regain lung function. Three steps are crucial in order to produce new and repair damaged elastin fibers in adults: (a) activation of tropo-elastin synthesis, (b) activation of the assembly of tropo-elastin proteins into polymeric chains, and (c) activation of lysyl oxidase-mediated crosslinking.

Compositions and methods for the treatment of lung emphysema and other forms of COPD are provided. These compositions comprise an active agent comprising copper, and a glycosaminoglycan or a physiologically acceptable salt thereof.

The compositions of the present invention are to be used to treat subjects suffering from, or at risk of developing lung emphysema, with or without air flow limitation, and other forms of COPD. Typically, the subject will be a mammal, in particular a human being but may be a vertebrate animal. The airflow limitation is usually both progressive and associated with reduced elasticity of the elastin fibers of the lung.

Methods of treating lung emphysema and other forms of COPD are provided. Such methods include diagnosing one or more disorders of the lung of a subject and administering a therapeutically effective amount of a composition comprising an active agent comprising copper, and a glycosaminoglycan or a physiologically acceptable salt thereof.

The term “treating” or “treatment” refers to executing a protocol, which may include administering one or more compositions or active ingredients to a patient (human or otherwise), in an effort to repair damaged lungs and/or prevent development of progression of the disease or disorder. “Treating” or “treatment” does not require complete halt of disease progression, does not require complete restoration of all lung damage, and specifically includes protocols which have only a marginal effect on the patient.

The term “therapeutically effective amount” means a quantity of the instant composition which, when administered to a patient, is sufficient to result in an improvement in patient's condition. The improvement does not mean a cure and may include only a marginal change in patient's condition. It also includes an amount of the active agents that prevents the condition or stops or delays its progression.

For the purposes of this invention “other forms of COPD” may be defined as a condition of airway wall thickening, bronchiectasis, chronic bronchitis and/or small airways disease.

The subject will typically be a mature adult. For example, the subject may be from 21 to 85, preferably from 25 to 70, more preferably from 30 to 60 and even more preferably from 40 to 50 years of age. The onset of any of, or a particular, symptom mentioned herein, will typically have been in adulthood. For example, the subject may have been at least 25, more preferably at least 30, still more preferably at least 35 and even more preferably at least 40 years of age before they experienced a particular symptom. In particular, the symptoms associated with more advanced stages of emphysema, such as any of those mentioned herein, may have their onset at such later stages of life. Subjects with a genetic predisposition to developing emphysema, such as those with alpha₁-antitrypsin deficiency, may develop the disease earlier. For example, they may display one or more, or a particular, symptom at from 20 to 31, preferably from 22 to 28, or more preferably from 24 to 26 years of age. Alternatively, they may first show the symptom at any of the age ranges mentioned herein. The subject may have been diagnosed at any of the ages, or within any of the age ranges, specified herein.

In cases where the subject is not human it may be a domestic animal or an agriculturally important animal. The animal may, for example, be a sheep, pig, cow, bull, poultry bird or other commercially farmed animal. In particular, the animal may be a cow or bull and preferably is a dairy cow. The animal may be a domestic pet such as a dog, cat, bird, or rodent. In a preferred embodiment the animal may be a cat or other feline animal. The animal may be a monkey such as a non-human primate. For example, the primate may be a chimpanzee, gorilla, or orangutan. In a preferred embodiment of the invention the animal may be a horse and, for example, may be a racehorse.

The main therapeutically active ingredients of the compositions of the present invention are copper and glycosaminoglycan. These ingredients will be discussed in more detail below.

### Copper

The compositions of the present invention employ an active agent comprising a copper compound. The term “active agent”, as used herein, refers to a chemical element of compound that has a stimulating effect on repair and development of pulmonary elastin. The active agent comprises a copper compound, in particular a copper salt. Various copper salts may provide a source for the copper compounds. Suitable copper salts include but are not limited to copper sulfate, copper chloride, copper gluconate, copper acetate, copper heptanoate, copper oxide, copper methionate, dicopper oxide, copper chlorophyliin, and calcium copper edetate. Of these, copper sulfate is preferred.

### Glycosaminoglycan

The compositions of the present invention employ glycosaminoglycans, and in particular heparin. Glycosaminoglycans are linear hetero-polysaccharides possessing characteristic disaccharide repeat sequences that are typically highly N- and O-sulphated at D-glucosamine, galactosamine and uronic acid residues.

Any suitable glycosaminoglycan may be employed in the invention. Glycosaminoglycans and glycosaminoglycan salts suitable for use in the present invention will have an average molecular weight of from 12 to 18 kd. The glycosaminoglycan or salt may be present in various molecular weight sizes within this range. For further details reference may made to the prior art, in particular WO 03/068187 and its counterpart EP 1 511 466, the contents of which are herein incorporated by reference.

The glycosaminoglycan may be any suitable commercially available glycosaminoglycan and may, for example, be an unfractionated glycosaminoglycan. The glycosaminoglycan will have typically been isolated from a natural sources such as from an animal. In some cases, the glycosaminoglycan may have been synthesized rather than be a naturally occurring molecule.

Any suitable physiologically acceptable glycosaminoglycan salt may be employed in the invention and in particular a metallic salt, for example a sodium salt, an alkali metal or an alkaline earth metal salt. Other salts include calcium, lithium and zinc salts. Ammonium salts may also be used. The salt may be a sodium glycosaminoglycanate or glycosaminoglycan sulphate. Salts of derivatives of specific glycosaminoglycans mentioned herein may also be used in the invention. In the present application where mention of a glycosaminoglycan is made, such mention also includes physiologically acceptable salts thereof.

In a particularly preferred embodiment of the invention the glycosaminoglycan employed will be any of chondroitin sulfates A to E heparin, heparin sulfate, heparan, heparan sulfate, hyaluronic acid, keratan sulfate, a derivative of any thereof or a physiologically acceptable salt thereof or a mixture or any two thereof.

Heparin is a naturally occurring mucopolysaccharide present in a variety of organs and tissues, particularly liver, lung, and the large arteries. Heparin is a polymer of alternating a-D-glucosamine and hexuronate residues joined by (1,4) glycosidic linkages.

When glycosaminoglycans are synthesized in nature, typically they are conjugated to a central protein core. However, preferably the glycosaminoglycans employed in the invention will lack such a central core. Commercially available preparations of glycosaminoglycans will usually lack the core and may be employed. Preferably, unfractionated heparin is used in the formulation. Instead of unfractionated heparin, low-molecular weight heparins, comprising dalteparin and enox-aparin, and other members of the glycosaminoglycan family, including heparan sulfate, could be used with the copper compound in the inhalation formulation to stimulate tropo-elastin polymerization and/or prevent copper-induced collagen crosslinking.

Heparin is clinically used as an anti-coagulant, where it is thought to exert its effects through interaction with anti-thrombin III (AT-Ill) and heparin co-factor II and other coagulation factors. Typically the heparin will retain some anticoagulant activity i.e. be able to increase clotting time in an individual. Thus, preferably the heparin will be able to bind anti-thrombin III (AT-Ill) and/or heparin co-factor II (HCII) and hence inhibit clotting. Preferably it will be able to form a complex with AT-Ill, thrombin and a clotting factor. However, in some embodiments a heparin which lacks anti-coagulant activity or which has reduced anti-coagulant activity may also be employed. Thus the heparin may have been modified so that it has from 0 to 80%, preferably from 5 to 60%, more preferably from 10 to 40% and even more preferably from 10 to 30% of the activity of the unmodified form or in comparison to unmodified heparin. Other glycosaminoglycans, in particular dermatan sulphate, also possess anticoagulant activity. Preferably, therefore, the glycosaminoglycans and their derivatives employed will retain some anti-coagulant activity, as discussed above for heparin and its derivatives.

### Other Components

For the reactivation of pulmonary elastin fiber production, repair of damaged elastin fibers, deceleration of the rate of elastin degradation and inhibition of advanced glycation end product (AGE) formation it may be of benefit to combine the composition of the invention comprising a copper compound and a glycosaminoglycan with other healthy or pharmaceutically active components in a single composition, or in the form of a kit for simultaneous, sequential or separate administration. For instance, it is envisaged that the composition of the invention could be provided in conjunction with medicaments or substances with effects on elastin metabolism in the vasculature, selected from the polyphenols epigallocatechin-(3-)gallate (EGCG) and pentagalloyl glucose (PGG), ATP-dependent potassium-channel openers, e.g. minoxidil, nicorandil, diazoxide, pinacidil, and cromakalin, magnesium, vitamin K1, vitamin K2, breakers of AGEs in arteries, e.g. amino-guanidine, pyridoxamine, N-phenacylthiazolium bromide, alagebrium, and flavonoids (e.g. kaempferol, genistein, quercitrin, quercetin, and epicatechin), compounds with potential effects on elastin metabolism in the lungs, selected from vitamin A, vitamin D and penta galloyl glucose.

### Subject Assessment

The present invention provides for compositions comprising an active agent comprising a copper compound. and a glycosaminoglycan or a salt thereof for use in facilitating repair and development of elastin fibers in lungs of patients with emphysema and preventing copper-induced stimulation of collagen crosslinking. The copper compound and glycosaminoglycan or salt used, the route of delivery and any of the other parameters of the composition and subject being treated may be the same as described herein for any of the other embodiments of the invention.

The compositions of the invention preferably induce an improvement in the condition of the subject and/or prevention/deceleration of disease progression. The compositions may therefore be used to manage a patient suffering from, or prone to, emphysema and/or other forms of COPD as defined herein. They may prevent, ameliorate, improve or cure the condition. They may slow down or arrest the progressive deterioration characteristic of emphysema and other forms of COPD or in some cases even cause some reversal of the deterioration. They may prevent, reduce or reverse one or more of the symptoms associated with emphysema and other forms of COPD. They preferably will also increase the feeling of wellbeing in the subject and their quality of life.

A composition of the invention preferably reduces, eliminates, or at least prevents further increase in one or more of:
accelerated decline in lung function parameters including but not limited to FEV₁ and diffusing capacity
damage to the structure of the lung

Treatment with the compositions of the invention may also mean that the ratio of FEV₁/FVC does not decline further, or is improved. For example, the ratio may be closer to that expected in a healthy subject.

The compositions may reduce pulmonary elastin degradation and facilitate pulmonary elastin repair. They may also have a preventing effect on the accumulation of collagen in emphysematous lungs.

The compositions of the invention may reduce the breakdown of the structure of the lung, such as the degradation of elastin in the airways and in the alveoli and hence the loss of lung elasticity. They may reduce or prevent the collapse of portions of the lung and/or the development of enlarged airspaces in which air can become trapped. The compositions may prevent or reduce any of the pathological changes associated with emphysema and other forms of COPD outlined herein. In particular, they may prevent progression of a pathological change. They may also prevent, or delay; the onset of a particular pathological change.

The compositions of the invention may typically reduce the decline in lung function parameters, such as diffusing capacity and FEV₁, by from 10 to 100%, preferably from 20 to 80%, more preferably from 30 to 60% and even more preferably from 40 to 50%. They may reduce the annual decline in FEV₁ by from 10 to 100 ml, preferably from 20 to 60 ml and even more preferably from 30 to 40 ml per year. In some cases on treatment the subject will display an improvement of lung function parameters so that FEV₁ and diffusing capacity are from 25 to 100%, preferably from 40 to 100%, more preferably from 60 to 100% and even more preferably from 80 to 100% of the predicted value.

Measurement of lung density with CT-scans is a convenient method to quantify the severity of lung emphysema. The compositions of the invention may slow down or arrest the progressive decline of CT-lung density in patients with emphysema or may even increase lung density.

The compositions of the invention may reduce lung tissue degradation and facilitate repair of damaged lung tissue.

The compositions of the invention may eliminate, delay the onset, or reduce the severity of any of the symptoms and features of emphysema and other forms of COPD mentioned herein.

Administration and Formulation

The pharmaceutical compositions of the present invention may be prepared by formulating the at least one copper compound, preferably copper sulfate, and the glycosaminoglycan, preferably heparin, with a standard physiologically, and in particular pharmaceutically, acceptable carrier and/or excipient as is routine in the pharmaceutical art.

The exact nature of the formulation will depend upon several factors including the particular copper compound and glycosaminoglycan employed and the desired route of administration. Suitable types of formulation are fully described in Remington's Pharmaceutical Sciences, 22nd Edition, Mack Publishing Company, Eastern Pa., USA, the disclosure of which is included herein in its entirety by way of reference.

In an especially preferred embodiment, the compositions comprising a copper compound and a glycosaminoglycan are administered as inhalation therapy including but not limited to inhaling a nebulization formulation, metered dose inhalers, or in a form suitable for a dry powder inhaler. The composition may be present in a blister pack or breakable capsule, Thus administration may typically be via the mouth.

As the compositions according to the invention will typically be administered via inhalation or via installation, preferably it will be in a form suitable for administration via such a route. In particular, the compositions may be in a form suitable for inhalation and/or installation.

Suitable methods for formulating and preparing the compositions to be administered via inhalation are well known in the art and may be employed in the present invention. The composition exemplified by copper sulfate and heparin as nebulization therapy can be used with excipients comprising saline. The composition as dry-powder formulation can be used with excipients comprising lactose. The composition in a metered dose inhaler can be used with excipients comprising propellants comprising hydrofluoro-alkane (HFA), co-solvents comprising ethanol, and stabilizers comprising oleic acid.

The necessary dose to be administered will normally be determined by a physician, but will depend upon a number of factors, such as the condition to be treated and the condition of the patient. Examples of doses and dose ranges will be given below. The preferred duration of administrations, the preferred frequency of administrations and the preferred dose of administrations depend on a variety of factors including but not limited to age, body weight and the severity of the emphysematous lesions quantified by CT lung densitometry measurements and lung function tests. The length of treatment may typically be from two weeks, a month, six months a year or more. In many cases the subject will remain on the compositions of the invention permanently or for extended periods. In patients with more severe forms of emphysema, the preferred duration of using the invention is life-long and the preferred frequency of administration is once daily. In milder forms of emphysema, a temporary period of administration and less frequent administrations than once daily may suffice.

The severity of copper deficiency in the lungs is another determinant of the treatment intensity. Copper measurement in exhaled breath condensate is a convenient method to calculate the copper deficit in the lungs in order to guide the intensity and duration of copper inhalation therapy.

The pharmaceutical compositions according to the present invention exemplified by copper sulfate and heparin are preferably and effectively administered in the following doses, depending inter alia on factors such as age, sex, body weight and condition of the patient. The preferred doses of both copper sulfate and heparin are derived from cell culture studies with fibroblasts described below (see the “Experimental” section), in which various doses and combinations are assessed for their effects on elastin repair and development.

(a) With regard to the copper salt, between 1 μg and 10 mg per day, preferably between 50 μg and 2 mg per day, more preferably between 100 μg and 1 mg per day and most preferably between 200 and 500 μg per day. These doses will typically be given once, twice or three times a day, preferably once a day.

(b) With regard to heparin, between 100 and 1,500,000 IU per day, preferably between 5,000 and 1,000,000 IU per day, more preferably between 25,000 and 500,000 IU per day and most preferably between 50,000 and 250,000 IU per day. A unit of heparin activity (United States Pharmacopeia) is defined as the amount of heparin that prevents 1 ml of citrated sheep plasma from clotting for one hour after adding 0.2 ml of 1% CaCl₂. These doses will typically be given once, twice or three times a day and will preferably be given once a day.

A preferred composition for inhalation contains about 0.5-1 mg copper sulfate and about 150,000 IU heparin.

The therapeutically active components which constitute a composition according to the present invention are preferably administered simultaneously, but may also be given sequentially or separately, if desired.

In some preferred embodiments, the compositions of the present invention may be formulated as aerosols. The formulation of pharmaceutical aerosols is routine to those skilled in the art, see for example, Sciarra, J. in Remington (supra). The agents may be formulated as solution aerosols, dispersion or suspension aerosols of dry powders, emulsions or semisolid preparations. The aerosol may be delivered using any propellant system known to those skilled in the art. The aerosols may be applied to the lower respiratory tract. The compositions comprising a copper compound and heparin may be delivered using liposomes and nanoparticle delivery methods which are known to a person skilled in the art. Liposomes, particularly cationic liposomes, may be used in carrier formulations.

The compositions for use in accordance with the present invention, may include, in addition to active ingredient, a pharmaceutically acceptable excipient, carrier, buffer, stabilizer or other materials well known to those skilled in the art. In particular they may include a pharmaceutically acceptable excipient. Such materials should be non-toxic and should not interfere with the efficacy of the active ingredient. The precise nature of the carrier or other material will depend on the route of administration. Suitable pharmaceutical carriers are described in Remington (supra).

The compositions of the present invention may be delivered by any device adapted to introduce one or more therapeutic compositions into the lower respiratory tract. In some preferred embodiments, the devices of the present invention may be metered-dose inhalers. The devices may be adapted to deliver the therapeutic compositions of the invention in the form of a finely dispersed mist of liquid, foam or powder. The device may use a piezoelectric effect or ultrasonic vibration to dislodge powder attached on a surface such as a tape in order to generate mist suitable for inhalation. The devices may use any propellant system known to those in the art including, but not limited to, pumps, liquefied-gas, compressed gas and the like.

In cases where the copper compound and heparin are administered in the form of particles or droplets, the particle/droplet size and/or other properties of the particle/droplet may be chosen to ensure that the particles are delivered to a particular region of the respiratory tract. For example, they may be designed to reach only the lower parts of the respiratory tract. In cases where the copper compound and heparin are delivered in an aqueous form preferably the solution will be isotonic to help ensure effective delivery to the subject. In particular, particles with a diameter of 10 μM are thought to be effective in reaching the lower parts of the respiratory tract and hence may be employed where such a site is the desired target for the compositions. In embodiments, where it is desired to deliver the composition to the lower parts of the respiratory tract, such as alveoli for example, the diameter of the particles administered may be less than 10 μM, preferably less than 8 μM, more preferably less than 6 μM and even more preferably less than 4 μM. In a preferred embodiment the particles may have a diameter of 3 μM or less and more preferably may have a diameter of 2 μM or less. In an especially preferred embodiment the particles will have a diameter of from 3 to 5 μM. In some cases the particles administered may be less than 1000 nm, preferably less than 500 nm, more preferably less than 250 nm and still more preferably less than 100 nm in diameter. The sizes may refer to particles of solid matter or droplets of solutions and suspensions.

The size of particles necessary to penetrate to a specific part of the respiratory tract will be known in the art and hence the particle size can be chosen to suit the target size. Techniques such as milling may be used to produce the very small particles necessary. In some cases the desired part of the respiratory tract may be the upper respiratory tract and hence larger particles sizes may be employed. The density of the particles and their shape may also be chosen to facilitate their delivery to the desired site.

The compositions of the invention may take a variety of forms. They may be in the form of powders, powder microspheres, solutions, suspensions, gels, nanoparticle suspensions, liposomes, emulsions or microemulsions. The liquids present may be water or other suitable solvents such as a CFC or HFA. In the case of solutions and suspensions these may be aqueous or involve solutions other than water.

Devices of the present invention typically comprise a container with one or more valves through which the flow of the therapeutic composition travels and an actuator for controlling the flow. Suitable devices for use in the present invention may be seen in, for example, Remington (supra). The devices suitable for administering the compositions of the invention include inhalers and nebulizers such as those typically used to deliver steroids to asthmatics. In some cases, a spacer may be used in conjunction with the inhaler to help ensure effective delivery.

Various designs of inhalers are available commercially and may be employed to deliver the compositions of the invention. These include the Accuhaler, Aerohaler, Aerolizer, Airmax, Autohaler, Breezhaler, Clickhaler, Diskhaler, East-breathe inhaler, Easyhaler, Evohaler, Ellipta, Fisonair, Handihaler, Integra, Jet inhaler, Miat-haler, Nexthaler, Novolizer inhaler, Pulvinal inhaler, Respimat, Rotahaler, Spacehaler, Spinhaler, Syncroner inhaler and Turbohaler devices. A number of formulation techniques which produce particularly desirable particles are known in the art and may be employed. For example the nanocrystal, pulmosol and pulmosphere technologies may be employed.

In some cases the compositions may be administered via installation. In such cases, typically the composition will be in liquid form and will be administered via an artificial airway such as, for example, an endotracheal tube. The liquid will typically be drawn up into a syringe and then expelled through the artificial away into the respiratory tract of the subject. Installation is often used in an emergency context. In many cases it may be used where the subject has a relatively advanced form of CAL and has been admitted to hospital.

The compositions may include various constituents to optimize their suitability for the particular delivery route chosen. The viscosity of the compositions may be maintained at a desired level using a pharmaceutically acceptable thickening agent. Thickening agents that can be used include methyl cellulose, xanthan gum, carboxymethyl cellulose, hydroxy-propyl cellulose, carbomer, polyvinyl alcohol, alginates, acacia, chitosans and combinations thereof. The concentration of the thickening agent will depend upon the agent selected and the viscosity desired.

In some embodiments, the compositions may comprise a humectant. This may help reduce or prevent drying of the mucus membrane and to prevent irritation of the membranes. Suitable humectants include sorbitol, mineral oil, vegetable oil and glycerol; soothing agents; membrane conditioners; sweeteners; and combinations thereof.

The compositions may comprise a surfactant. Suitable surfactants include nonionic, anionic and cationic surfactants. Examples of surfactants that may be used include, for example, polyoxyethylene derivatives of fatty acid partial esters of sorbitol anhydrides, such as for example, Tween 80, Polyoxyl 40 Stearate, Polyoxy ethylene 50 Stearate, fusicates, bile salts and Octoxynol.

The synergistic effects of the compositions according to the present invention comprising an active agent comprising a copper compound, and a glycosaminoglycan in inhalation therapy, exemplified by copper sulfate and heparin, respectively, will be further demonstrated in the “Experimental” section.

### Experimental

The studies, on which the present invention is based, are part of a research project following a systematic approach with the objective to establish specific therapy for patients with lung emphysema.

The focus of this project is on the pulmonary extracellular matrix macroproteins elastin and collagen, as well as other proteins with crucial roles in the development and repair processes of elastin and collagen fibers: i.e. tropo-elastin, fibulin-4, fibulin-5, “prototype” LOX, and LOXL1.

The level of elastin crosslinking was quantified by measuring the elastin-specific crosslinking amino acids desmosine and isodesmosine (together referred to as DES) [3], and the level of collagen crosslinking was quantified by measuring the collagen-specific crosslinking amino acid hydroxyproline.

In applicant's research project, experiments were undertaken in the following sequential order:
1. Histological examination of lung biopsies from patients with emphysema, idiopathic pulmonary fibrosis (IPF), combined pulmonary fibrosis and emphysema (CPFE) and control subjects with no parenchymal lung disease.
2. Staining of lung biopsies with anti-active-LOXL1 and LOXL2 antibodies.
3. Gene expression studies on lung tissues from patients with emphysema, IPF, CPFE and control subjects with no parenchymal lung disease.
4. Measurements of copper levels in exhaled breath condensate of patients with emphysema and IPF and controls with no lung disease.
5. Measurements of copper levels in lung tissues of patients with emphysema, IPF, CPFE and control subjects with no parenchymal lung disease.
6. Cell cultures with pulmonary rat fibroblasts.
7. Repair mechanisms in a porcine pancreatic protease-induced emphysema model in mice.
8. Analyses of the nebulization of heparin sodium and copper sulfate solutions using a laser diffraction analysis method.

### 1. Histological Examination of Lung Biopsies

Rationale: We started this project with the examination of extracellular matrices from patients with emphysema, IPF, CPFE and control subjects. The reason for also studying lungs of fibrotic patients was our expectation that elucidating the so-called “divergence factor” in the pathogeneses of IPF and emphysema may help to establish the defect responsible for the unsuccessful elastin repair process in emphysematous lungs and to facilitate the establishment of specific therapy for patients with emphysema.

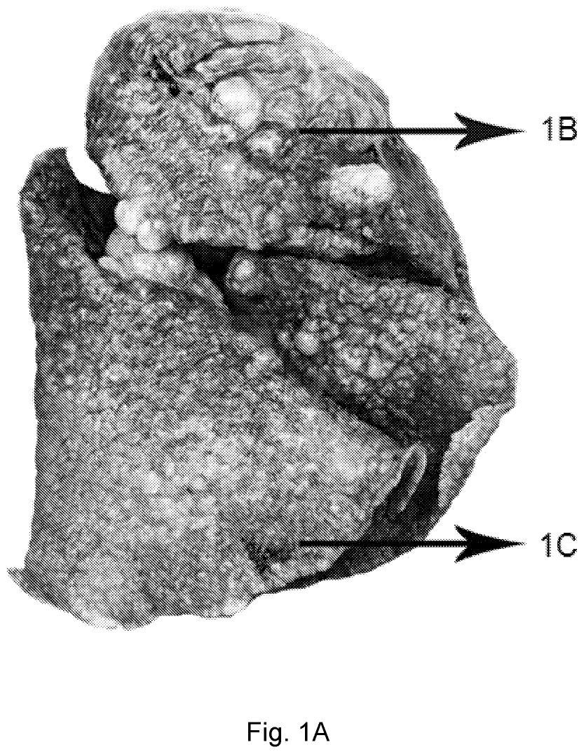

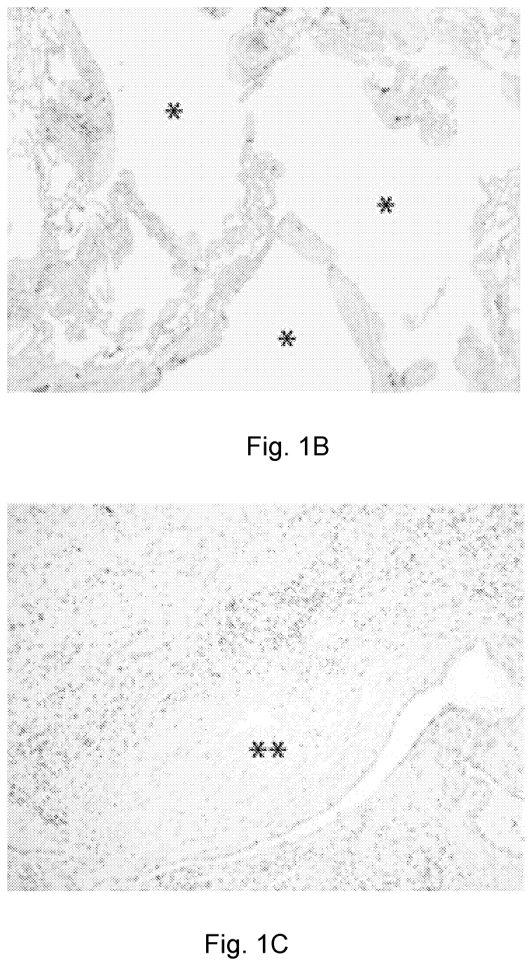

Methods: Lung tissue was obtained from surgical lung resection specimens in patients with emphysema (n=10) and healthy controls without COPD/emphysema (n=10); tumor-free lung tissue in the subpleural area at appropriate distance from the tumor was taken. Lung tissue was obtained from diagnostic surgical lung biopsies in patients with IPF (n=10). Lung tissues from apical (emphysematous) and basal (fibrotic) lung areas from patients with CPFE were obtained from explant lungs (n=4; FIG. 1). We first examined the pulmonary extracellular matrices with histological analyses: Masson's trichome stain for collagen and Verhoeff-Van Gieson stain for elastin.

Results: We found that the elastin content was reduced in lung parenchyma of patients with lung emphysema and increased in patients with IPF (FIG. 2). Collagen content was increased in both patients with emphysema and IPF compared to control lungs; however, we observed a more pronounced increase of collagen fibers in IPF. In patients with CPFE, elastin content was increased in the basal fibrotic lung parenchyma and reduced in the apical emphysematous lung parenchyma. Collagen content in patients with CPFE was increased in both apical emphysematous and basal fibrotic lung parenchyma, but was more pronounced in basal fibrotic lung areas. DES levels were reduced in emphysematous lungs and increased in IPF lungs. Hydroxyproline levels were increased in both emphysema and IPF lungs but much higher in the latter. Remarkably, the relative difference of collagen levels between emphysema and IPF lungs was much lower than the relative difference of hydroxyproline levels between emphysema and IPF lungs, indicating that collagen is less extensively crosslinked in emphysematous compared to IPF lungs.

Conclusions: From these analyses on fibrotic and emphysematous lungs, we concluded that our specific therapy for patients with emphysema should not only stimulate elastin fiber repair and development but should also inhibit collagen maturation, organization and accumulation, as collagen is abundantly present in emphysematous lungs.

### 2. Staining of Lung Biopsies for Active-LOXL1 and Active-LOXL2

Rationale: LOX enzymes are not only responsible for the crosslinking of tropo-elastin precursors into durable elastin fibers, but they also crosslink procollagen precursors into durable collagen fibers. Whereas elastin fibers provide elasticity, resilience, and deformability, collagen fibers provide tensile strength to the lungs. Excessive collagen deposition is a hallmark of lung fibrosis. Stimulation of lung fibrosis formation would be an unwanted side effect of LOX stimulation. We hypothesized that LOX enzymes would be decreased in emphysema and increased in fibrosis.

Methods: We stained same lung biopsies as used for the histological analysis with active-LOXL1 (Novus Biologicals; NBP1-82827) and active-LOXL2 (Novus Biologicals; NBP1-32954) antibodies.

Results: Compared to control subjects, the intensity of both active-LOXL1 and active-LOXL2 staining was enhanced in IPF patients and reduced in emphysema patients. In CPFE patients, the intensity of active-LOXL1 and active-LOXL2 stainings were enhanced in basal fibrotic lung parenchyma and reduced in apical emphysematous lung parenchyma.

### 3. Gene Expression Analyses in Lung Tissues

Rationale: We proceeded our systematic research project by gene expression (quantitative real-time polymerase chain reaction; qRT-PCR) analysis in lungs of patients with emphysema, IPF and CPFE compared to lungs of controls, in order to identify those elastin repair genes/proteins that are insufficiently upregulated in emphysema and should be stimulated to achieve efficacious elastin repair.

Methods: We analyzed expression of the following genes in the previously mentioned lung biopsies: tropo-elastin (ELN), LOX, LOXL1, LOXL2, fibulin-4 and fibulin-5.

Results: To our surprise, we found that ELN and fibulin-5 were strongly upregulated in both patients with emphysema and IPF, suggesting that these proteins are not the “divergence factor” between lung emphysema and fibrosis. LOXL1 was upregulated in IPF patients compared to controls. No significant difference in LOXL1 gene expression was found between patients with emphysema and control subjects.

Conclusions: We concluded from the gene expression study that stimulation of ELN and fibulin-5 syntheses may not be essential targets of therapy for pulmonary elastin repair, since these proteins are already upregulated in lungs of patients with emphysema.

### Interim Analysis

We were confronted with the paradox that activated-LOXL1 levels were reduced in emphysematous lungs; however, expression of LOXL1 was not reduced in the qRT-PCR. Based on the interim analysis of the results from our systematic research project, we concluded that patients do not develop emphysema because of decreased levels of the protein LOXL1, and hypothesized that patients may develop emphysema because of decreased levels of LOXL1's essential cofactor, i.e. copper. We conducted several studies to test this hypothesis.

### 4. Copper in Exhaled Breath Condensate and Serum

Rationale: As copper in an essential cofactor for the activation of LOX enzymes, we hypothesized that copper concentrations would be decreased in patients with emphysema.

Methods: At first, we measured copper levels in serum of patients with emphysema (n=10) and controls (n=10). Subsequently, we collected exhaled breath condensate (EBC) with the RTube™ (Respiratory Research; www.repiratoryresearch.com) and measured copper levels.

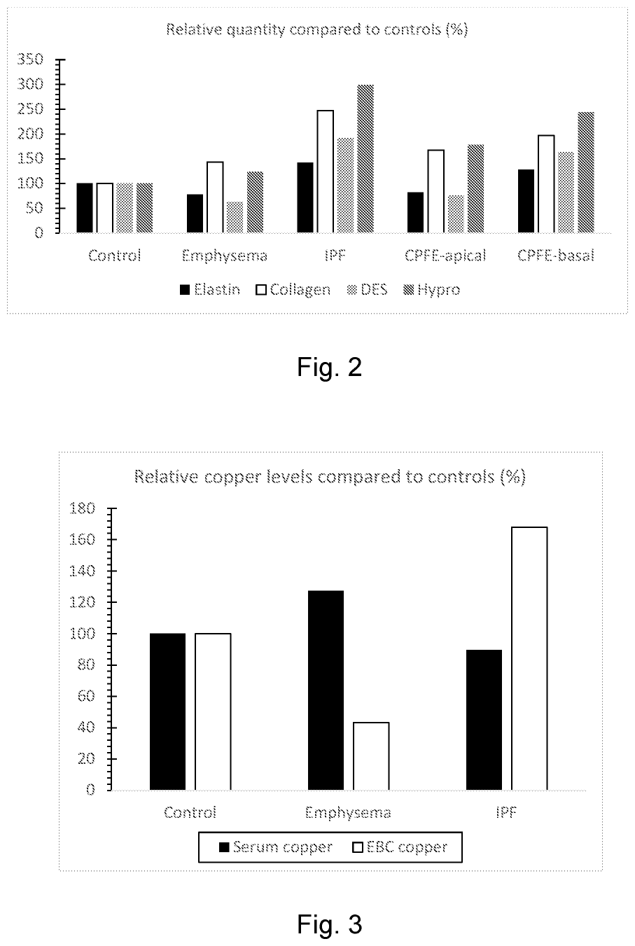

Results: In contrast to our hypothesis, serum copper levels were not reduced but increased in patients with emphysema (FIG. 3). EBC copper concentrations, however, were reduced in emphysema patients compared to controls.

Conclusions: There is a local pulmonary and no systemic copper deficiency in emphysema.

### 5. Copper Concentrations in Lung Biopsies

Rationale: As copper is an essential cofactor for the activation of LOX enzymes, we hypothesized that copper concentrations would be decreased in patients with emphysema and increased in patients with IPF.

Methods: We measured copper concentrations in lung parenchyma of patients with emphysema, IPF and CPFE.

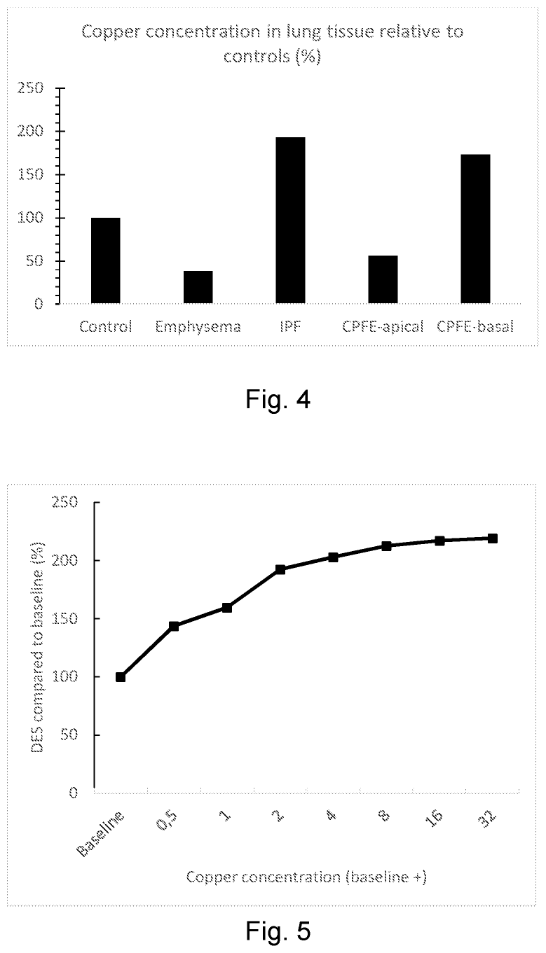

Results: We found that copper concentrations were indeed reduced in emphysema and increased in fibrosis compared to control lungs (FIG. 4). We also found a strong gradient in copper concentrations between the apical emphysematous (low copper levels) and basal fibrotic (high copper levels) parenchyma in lungs of CPFE patients. Our explanation for these surprising differences in copper concentrations within CPFE lungs is that copper delivery to the upper lung zones is much lower than to the lower lung zones, which seems logical given that apical lung zones are very poorly perfused.

Conclusions: The copper inhalation therapy is to be preferred over systemic routes of administration, (a) as the lung apices are far better ventilated than perfused and (b) as there is local and no systemic copper deficiency. There is also a third important reason to prefer inhaled copper therapy above oral administration. Serum copper levels are positively associated with the risk for developing Alzheimer's disease [38]. In order to achieve the same concentrations of copper in the lungs (particularly in the apical lung zones), much lower doses of copper are needed with inhalation therapy than with oral therapy. We intratracheally administered copper to mice and indeed found no effect of this intervention on cerebral copper concentrations.

The high copper concentrations in lung parenchyma of patients with IPF and in the fibrotic basal lung areas of patients with CPFE formed the basis for our apprehension that stimulating the activation of LOX enzymes by copper inhalation therapy will stimulate collagen crosslinking and thereby collagen maturation/organization, since LOX enzymes are not only a crosslinkers of elastin but also of collagen [39]. Copper-induced accumulation of collagen in emphysematous lung would be deleterious, as (a) collagen levels are already increased in emphysematous lungs and (b) it may cause the transition from emphysema to fibrosis (i.e. transition of one devastating lung disease into the other).

Therefore, we concluded that we should combine copper with one or more other ingredients in our inhalation formulation to prevent copper-induced accumulation of collagen.

6a. Fibroblast Cell Culture with Additional Copper

Rationale: Based on copper deficiency as the most likely cause of inefficacious elastin repair process in patients with emphysema, we hypothesized that copper supplementation would stimulate the elastin development/repair process by activating more LOX enzymes.

Methods: Fibroblasts were grown for 21 days after which they were lysed and mRNA was extracted. Medium was replenished twice a week. qPCR was performed to measure the expression of LOX, LOXL1 and elastin (ELN coding for tropo-elastin) genes. LOX activity was measured using Amplite Fluorimetrix LOX Assay Kit (AAT Bioquest, Sunnyvale, Calif., USA). Total insoluble elastin deposited in the cell layers and soluble tropo-elastin were measured with the FastinTM Elastin assay kit (Biocolor, UK). DES levels were measured using liquid chromatography-tandem mass spectrometry method in the Canisius-Wilhelmina Hospital (Nijmegen, The Netherlands), as previously described [9]. Collagen in the medium and matrix were quantified using Sircol™ INSOLUBLE Collagen Assays (Biocolor, UK). We first measured copper levels in the fibroblast medium. Subsequently, we added additional copper sulfate in ascending concentrations, i.e. +0.5*initial copper concentration in the fibroblast medium, +1*initial copper concentration, +2*initial copper concentration, +4*initial copper concentration, +8*initial copper concentration, +16*initial copper concentration and +32*initial copper concentration, in order to make dose-response between copper concentration and the other variables.

Results: Copper sulfate increased LOX and LOXL1 gene expression, LOX activity, DES levels (all favorable; FIG. 5) as well as insoluble collagen levels (unfavorable) in a dose-dependent manner. Copper sulfate did not have any effect on ELN gene expression.

Conclusions: Adding additional copper sulfate to the cell culture medium had a favorable stimulating effect on the accumulation of crosslinked elastin fibers; however, it also had an unfavorable stimulating effect on the accumulation of insoluble collagen levels. The dose response curve, with regard to DES levels, topped of at a copper concentration of about +8*initial copper concentration in the fibroblast medium (FIG. 5).

6b. Fibroblast Cell Cultures to Test Potential Synergistic Effects of Retinoic Acid, Minoxidil and Heparin on Top of Copper Sulfate

Rationale: In the second part of the cell culture studies, we assessed whether addition of other substances to copper sulfate would further stimulate the development/repair process of elastin.

Methods: We added retinoic acid, minoxidil and heparin to copper-enriched fibroblast medium (copper concentration of +8*initial copper concentration in the fibroblast medium).

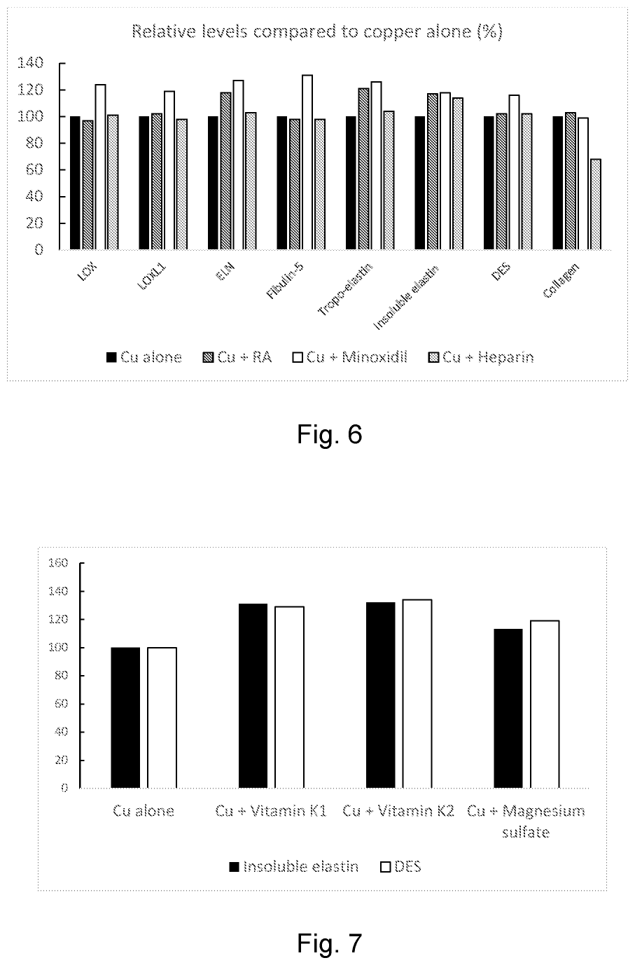

Results (FIG. 6): In contrast to copper sulfate monotherapy, addition of retinoic acid to copper sulfate had a stimulating effect on ELN gene expression and tropo-elastin levels. Addition of retinoic acid to copper sulfate also had an additional stimulating effect on insoluble elastin levels; however, retinoic acid had no additional effect on DES levels. Retinoic acid had no additional effect to copper sulfate monotherapy on LOX and LOXL1 gene expression. Addition of minoxidil to copper sulfate had a stimulating effect on LOX, LOXL1, ELN and fibulin-5 gene expression. Addition of minoxidil to copper sulfate had an additional stimulating effect on tropo-elastin, insoluble elastin and DES levels compared to copper sulfate monotherapy. Addition of minoxidil to copper sulfate had no additional stimulating effect on the accumulation of collagen compared to copper sulfate monotherapy; however, addition of minoxidil neither had a suppressing effect on collagen accumulation. Addition of heparin to copper sulfate had no additional effect compared to copper sulfate monotherapy on either LOX, LOXL1, ELN and fibulin-5 gene expression; and no effect on tropo-elastin levels. Addition of heparin to copper sulfate had a small stimulating effect on total insoluble elastin levels compared to copper sulfate monotherapy; however, it did not have an additional effect on DES levels. Much more importantly and surprisingly, addition of heparin to copper sulfate had a strong suppressing effect on collagen accumulation compared to copper sulfate monotherapy.

Conclusions: Addition of retinoic acid, minoxidil and heparin had some additional effects on elastin development and repair processes compared to copper sulfate monotherapy. Surprisingly, adding heparin to copper sulfate had a strong inhibiting effect on collagen levels. We concluded from this study that heparin seems to be the ideal adjunct to copper for treating patients with emphysema to prevent copper-induced collagen accumulation.

6c. Fibroblast Cell Cultures to Test Potential Synergistic Effects of Vitamin K and Magnesium Sulfate on Top of Copper Sulfate

Rationale: In the third part of the cell culture studies, we assessed whether addition of other substances to copper sulfate would inhibit the rate of elastin degradation.

Methods: We added vitamin K1, vitamin K2 and magnesium sulfate to copper-enriched fibroblast medium (copper concentration of +8*initial copper concentration in the fibroblast medium).

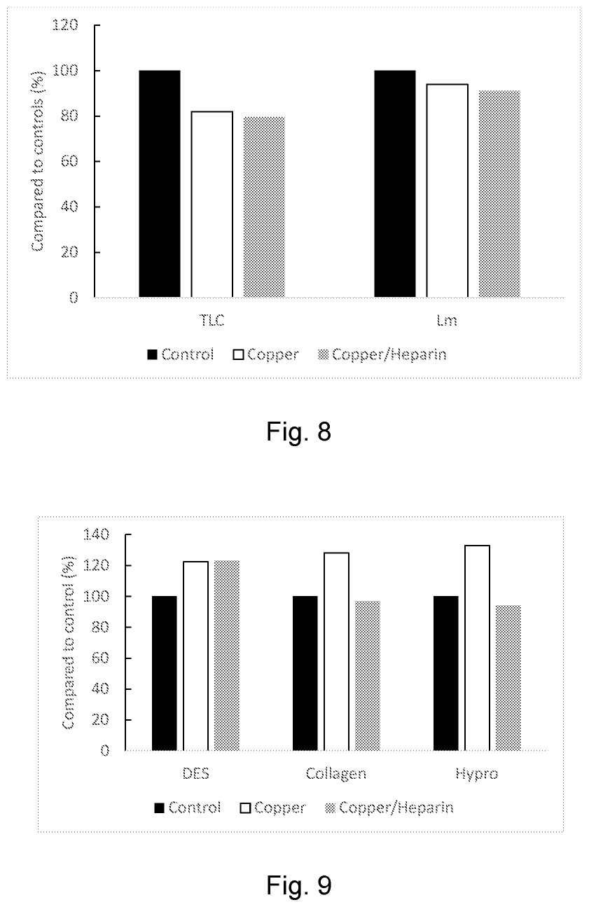

Results: Addition of vitamin K1, K2 and magnesium sulfate to copper sulfate had no stimulating effect on ELN gene expression, tropo-elastin levels or on LOX and LOXL1 gene expression. However, addition of vitamin K1, K2 and magnesium sulfate to copper sulfate had an additional stimulating effect on insoluble elastin levels and DES accumulation (FIG. 7). Addition of vitamin K1, vitamin K2 and magnesium sulfate to copper sulfate had no additional stimulating effect on the accumulation of collagen compared to copper sulfate monotherapy; however, addition of vitamin K1, vitamin K2 and magnesium sulfate neither had a suppressing effect on collagen accumulation.

Conclusions: Addition of vitamin K1, vitamin K2 and magnesium sulfate had additional effects on elastin and DES accumulation compared to copper sulfate mono-therapy. The most plausible mechanistic reason for this effect is an inhibitory effect of vitamin K1, vitamin K2 and magnesium sulfate on elastin degradation, as vitamin K1, vitamin K2 and magnesium sulfate did not have any effect on the elastin development processes. We concluded from this study that vitamin K1, vitamin K2 and magnesium sulfate seem to be useful adjuncts to copper for treating patients with emphysema to inhibit the rate of elastin degradation.

### 7. Emphysema Induced by Intratracheal Administration of Porcine Pancreatic Elastase

Rationale: Based on the very promising effects on both elastin and collagen metabolism of adding heparin to copper sulfate in the cell culture studies, we further assessed these effects in an animal model of emphysema.

Methods: In order to assess the effects of copper sulfate plus heparin on both elastin and collagen metabolism in vivo, we used a porcine pancreatic elastase (PPE)-induced emphysema model. Study was conducted in male BALB/c mice aged 7 weeks with a starting body weight of about 25 g. During the study period, all mice were housed in a conventional animal house with a 12/12 h light-dark cycle in filter-top cages and supplied with pelleted food and water ad libitum. 1.5 U porcine pancreatic elastase in 25 μL saline was intratracheally administered on day 1 under light anesthesia. 25 μL of either copper sulfate monotherapy (12.5 μg in 25 μL saline; n=4), a combination of copper sulfate (12.5 μg in 12.5 μL saline)/heparin (1,000 IU in 12.5 μL saline; n=4) or placebo (25 μL saline; n=4) was intratracheally administered under light anesthesia on day 1, 8, 15, 22 and 29. On day 35, mice were anesthetized intraperitoneally with a mixture of xylazine (8.5 mg/kg) and ketamine (130 mg/kg), they were tracheotomized and placed in a whole-body plethysmograph to assess lung function. After lung function measurements, mice were euthanized by an intracardiac administration of pentobarbital. The left lung will be snap-frozen in liquid nitrogen and stored at −80° C. for subsequent gene expression studies where ELN, LOX and LOXL1 were measured. The right lung was fixed in 6% paraformaldehyde at a constant hydrostatic pressure of 25 cm fluid column for 24 h. After dehydration and embedding in paraffin, sagittal sections will be stained with various stains and used for histological analyses to measure airspace enlargement (mean linear intercept); and subsequently this lung was used for measuring concentrations of both DES and insoluble collagen. Brains were taken out to measure copper concentrations.

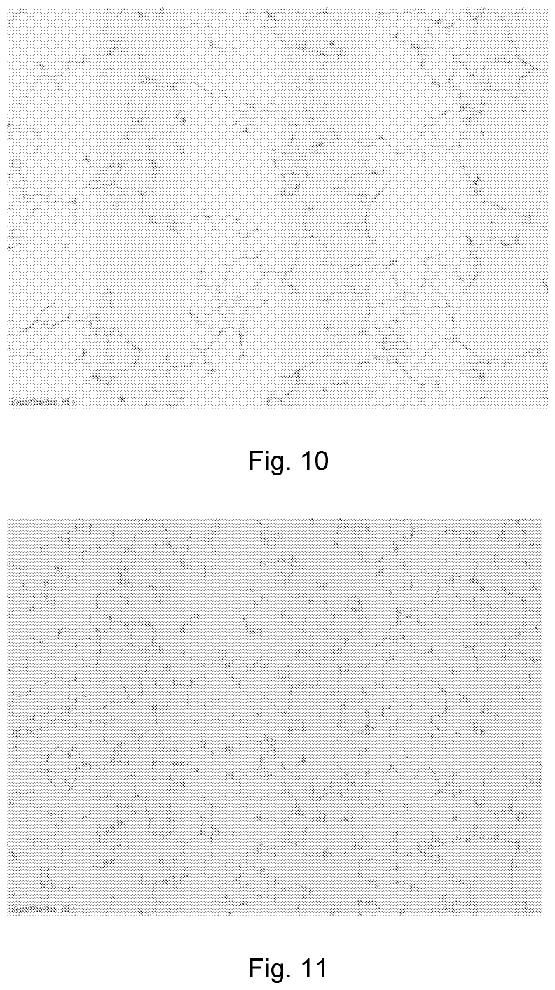

Results: More hyperinflation was present in the lung function tests of mice from the placebo group than from the copper sulfate and copper sulfate/heparin groups (FIG. 8). DES levels in lung tissue were higher in mice who received copper sulfate and copper sulfate/heparin than in those who received placebo (FIG. 9). Mean linear intercept was lower in mice who received copper sulfate and copper sulfate/heparin than in those who received placebo (FIGS. 8, 10 and 11). Insoluble collagen and hydroxyproline levels were elevated in mice who received copper sulfate monotherapy compared to placebo (FIG. 9). Insoluble collagen and hydroxyproline levels were significantly lower in mice who received copper sulfate/heparin compared to mice who received copper sulfate monotherapy and a little lower compared to mice who received placebo. Heparin is well-known as an anticoagulant; however, intratracheally administered heparin did not have any effect on systemic coagulation in mice.

Conclusions: We found that copper sulfate very effectively stimulated the elastin repair process but also induced accumulation and maturation of collagen fibers in the lungs. The combination of copper sulfate plus heparin very effectively facilitated repair of damaged elastin fibers (even better than copper sulfate monotherapy), and we found that, in contrast to copper sulfate monotherapy, copper sulfate plus heparin did not lead to an accumulation of collagen. Collagen and hydroxyproline levels were even lower after treatment with copper sulfate/heparin than after treatment with placebo. Inhaled heparin is thereby the ideal compound as adjuvant to the inhalation formulation with copper in order to prevent copper-induced collagen accumulation and to stimulate the elastin repair process.

### 8. Analyses of the Nebulization of Heparin Sodium and Copper Sulfate Solutions Using a Laser Diffraction Analysis Method

Rationale: It is essential to know whether it is feasible to nebulize a solution consisting of both heparin and copper with a commonly used nebulizer system, and whether this results in an adequate percentage of particles <5 μm.

Methods 1: We started our nebulization experiments with relatively low concentrations of copper and heparin. 26 mg heparin sodium (191 IU/mg) was solved in 1 mL sodium chloride 0.9%, and 12.5 mg copper sulfate (5 mg copper) was solved in 10 mL sodium chloride 0.9% of which 1 mL was used. 3 mL sodium chloride 0.9% was added to 1 mL heparin sodium (5,000 IU) solution and 1 mL copper sulfate (0.5 mg copper) solution. The 5 mL of nebulizing solution was loaded into a reusable nebulizer (PARI LC® Plus) and nebulized with a compressor (PARI BOY® SX). The aerosol was analyzed every 30 seconds using laser diffraction analysis (LDA) until the nebulizer started to sputter.

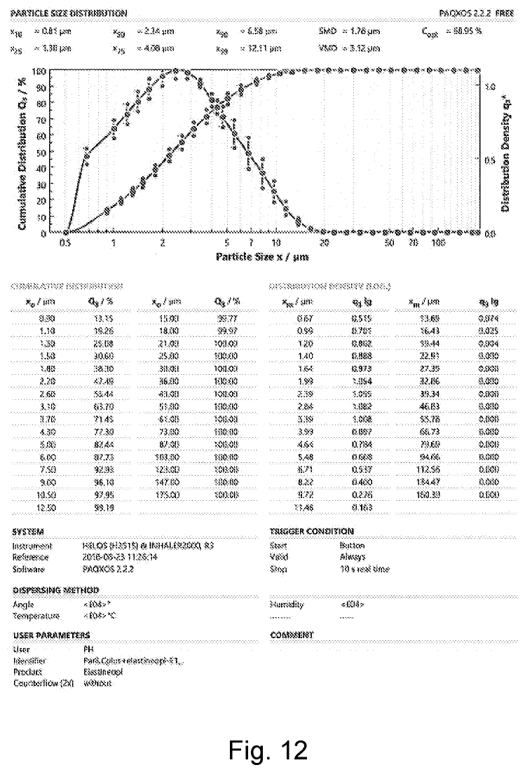

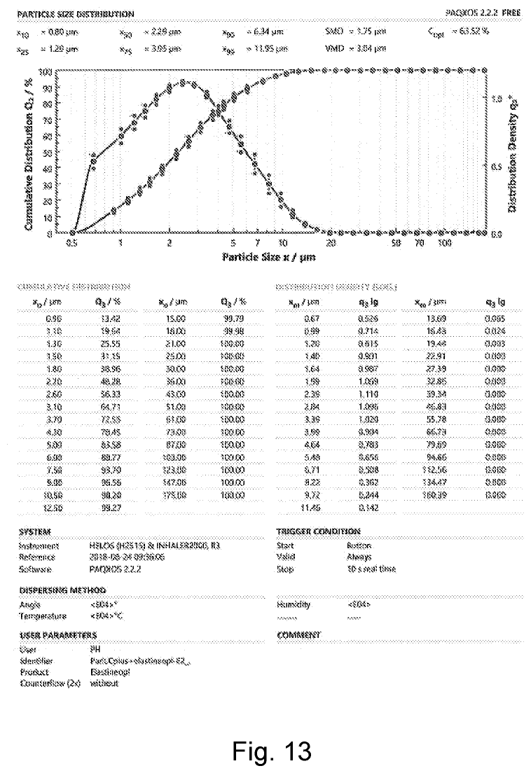

Results 1: The nebulizing time was about 3 minutes. The X₁₀ was 0.81 μm, the X₅₀ was 2.34 μm and the X₉₀ was 6.58 μm. The percentage of particles <5 μm was 82.44% (FIG. 12). A duplicate measurement of experiment 1 was conducted: nebulizing time was about 3 minutes, the X₁₀ was 0.80 μm, the X₅₀ was 2.29 μm, the X₉₀ was 6.34 μm and the percentage of particles <5 μm was 83.58% (FIG. 13).

Methods 2: 100,000 IU heparin sulfate and 1 mg copper were combined in a solution and sodium chloride 0.9% was added for a total volume of 5 mL.

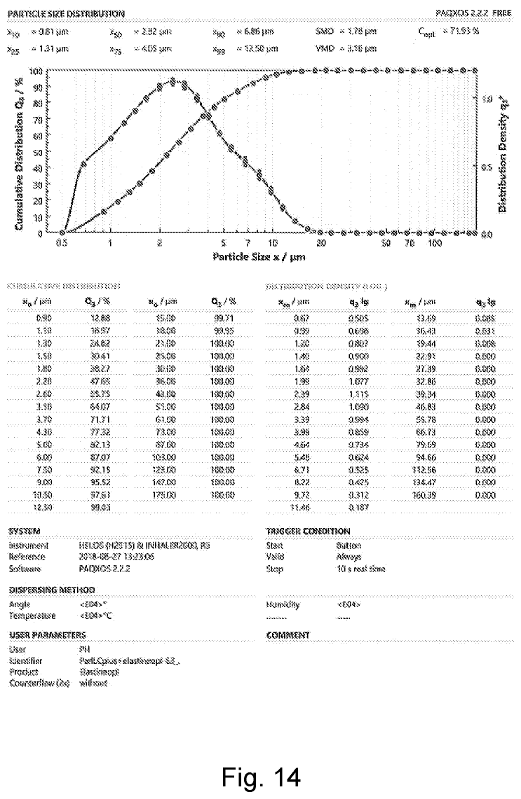

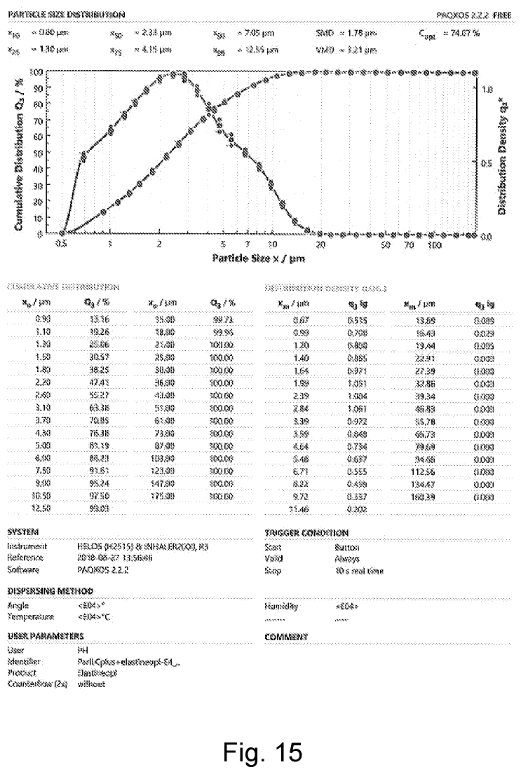

Results 2: The nebulizing time was about 4 minutes. The X₁₀ was 0.80 μm, the X₅₀ was 2.32 μm and the X₉₀ was 6.86 μm (FIG. 14). The percentage of particles <5 μm was 82.13%. A duplicate measurement of experiment 2 was conducted: nebulizing time was about 5 minutes, the X₁₀ was 0.80 μm, the X₅₀ was 2.33 μm, the X₉₀ was 7.05 μm and the percentage of particles <5 μm was 81.19% (FIG. 15).

Conclusions: It is feasible to combine heparin and copper in a nebulizing formulation and use of a commonly used nebulizer system results in a high percentage of particles <5 μm which would efficaciously reach the targeted alveolar areas in human lungs.

### LIST OF REFERENCES

1. From the Global Strategy for the Diagnosis, Management and Prevention of COPD, Global Initiative for Chronic Obstructive Lung Disease (GOLD) 2017. Available from: http://goldcopd.org.
2. Finlay G A, O'Donnell M D, O'Connor C M, et al. Elastin and collagen remodeling in emphysema. A scanning electron microscopy study. Am J Pathol. 1996; 149:1405-15.
3. Turino G M, Ma S, Lin Y Y, et al. Matrix elastin: a promising biomarker for chronic obstructive pulmonary disease. Am J Respir Crit Care Med. 2011; 184:637-41.
4. Mithieux S M, Weiss A S. Elastin. Adv Protein Chem. 2005; 70:437-61.
5. Umeda H, Aikawa M, Libby P. Liberation of desmosine and isodesmosine as amino acids from insoluble elastin by elastolytic proteases. Biochem Biophys Res Commun. 2011; 411:281-6.
6. Bostancioglu K, Mecham R P, Wallach J M. Elastolysis of normal and partially cross-linked elastin. Biochem Int. 1987; 15:263-9.
7. Tinker D, Romero-Chapman N, Reiser K, et al. Elastin metabolism during recovery from impaired crosslink formation. Arch Biochem Biophys. 1990; 278:326-32.
8. Liu X, Zhao Y, Gao J, et al. Elastic fiber homeostasis requires lysyl oxidase-like 1 protein. Nat Genet. 2004; 36:178-82.
9. Ma S, Lin Y Y, Cantor J O, et al. The Effect of Alpha-1 Proteinase Inhibitor on Biomarkers of Elastin Degradation in Alpha-1 Antitrypsin Deficiency: An Analysis of the RAPID/RAPID Extension Trials. Chronic Obstr Pulm Dis. 2016; 4:34-44.
10. Eurlings I M, Dentener M A, Cleutjens J P, et al. Similar matrix alterations in alveolar and small airway walls of COPD patients. BMC Pulm Med. 2014; 14:90.
11. Kranenburg A R, Willems-Widyastuti A, Moori W J, et al. Enhanced bronchial expression of extracellular matrix proteins in chronic obstructive pulmonary disease. Am J Clin Pathol. 2006; 126:725-35.
12. Tinker D, Geller J, Romero N, et al. Tropoelastin production and tropoelastin messenger RNA activity. Relationship to copper and elastin cross-linking in chick aorta. Biochem J. 1986; 237:17-23.
13. Maclay J D, McAllister D A, Rabinovich R, et al. Systemic elastin degradation in chronic obstructive pulmonary disease. Thorax. 2012; 67:606-12.
14. Mahoney M G, Brennan D, Starcher B, et al. Extracellular matrix in cutaneous ageing: the effects of 0.1% copper-zinc malonate-containing cream on elastin biosynthesis. Exp Dermatol. 2009; 18:205-11.
15. O'Dell B L, Kilburn K H, McKenzie W N, et al. The lung of the copper-deficient rat. A model for developmental pulmonary emphysema. Am J Pathol. 1978; 91:413-32.
16. Soskel N T, Watanabe S, Sandberg L B. Mechanisms of lung injury in the copper-deficient hamster model of emphysema. Chest. 1984; 85(6 Suppl):70S-73S.
17. Lucey E C, Keane J, Kuang P P, et al. Severity of elastase-induced emphysema is decreased in tumor necrosis factor-alpha and interleukin-1 beta receptor-deficient mice. Lab Invest. 2002; 82:79-85.
18. Vuillemenot B R, Rodriguez J F, Hoyle G W. Lymphoid tissue and emphysema in the lungs of transgenic mice inducibly expressing tumor necrosis factor-alpha. Am J Respir Cell Mol Biol. 2004; 30:438-48.
19. Liu L, Geng X, McDermott J, et al. Copper Deficiency in the Lungs of TNF-α Transgenic Mice. Front Physiol. 2016; 7:234.
20. McDonald F J. COMMD1 and ion transport proteins: what is the COMMection? Focus on “COMMD1 interacts with the COOH terminus of NKCC1 in Calu-3 airway epithelial cells to modulate NKCC1 ubiquitination”. Am J Physiol Cell Physiol. 2013; 305:C129-30.
21. Besiktepe N, Kayalar O, Ersen E, et al. The copper dependent-lysyl oxidases contribute to the pathogenesis of pulmonary emphysema in chronic obstructive pulmonary disease patients. J Trace Elem Med Biol. 2017; 44:247-55.
22. Mutti A, Corradi M, Goldoni M, et al. Exhaled metallic elements and serum pneumoproteins in asymptomatic smokers and patients with COPD or asthma. Chest. 2006; 129:1288-97.
23. Grange D K, Kaler S G, Albers G M, et al. Severe bilateral panlobular emphysema and pulmonary arterial hypoplasia: unusual manifestations of Menkes disease. Am J Med Genet A. 2005; 139A:151-5.
24. Shute J K, Calzetta L, Cardaci V, et al. Inhaled nebulised unfractionated heparin improves lung function in moderate to very severe COPD: A pilot study. Pulm Pharmacol Ther. 2018; 48:88-96.
25. Shute J K, Puxeddu E, Calzetta L. Therapeutic use of heparin and derivatives beyond anticoagulation in patients with bronchial asthma or COPD. Curr Opin Pharmacol. 2018; 40:39-45.
26. Setozaki S, Minakata K, Masumoto H, et al. Prevention of abdominal aortic aneurysm progression by oral administration of green tea polyphenol in a rat model. J Vasc Surg. 2017; 65:1803-12.
27. lsenburg J C, Simionescu D T, Starcher B C, et al. Elastin stabilization for treatment of abdominal aortic aneurysms. Circulation. 2007; 115:1729-37.
28. Sinha A, Nosoudi N, Vyavahare N. Elasto-regenerative properties of polyphenols. Biochem Biophys Res Commun. 2014; 444:205-11.
29. Barber T, Esteban-Pretel G, Marin M P, et al. Vitamin a deficiency and alterations in the extracellular matrix. Nutrients. 2014; 6:4984-5017.
30. Frankenberger M, Hauck R W, Frankenberger B, et al. All trans-retinoic acid selectively down-regulates matrix metalloproteinase-9 (MMP-9) and up-regulates tissue inhibitor of metalloproteinase-1 (TIMP-1) in human bronchoalveolar lavage cells. Mol Med. 2001; 7:263-70.
31. Brooks A D, Tong W, Benedetti F, et al. Inhaled aerosolization of all-trans-retinoic acid for targeted pulmonary delivery. Cancer Chemother Pharmacol. 2000; 46:313-8.
32. Frankenberger M, Heimbeck I, Moller W, et al. Inhaled all-trans retinoic acid in an individual with severe emphysema. Eur Respir J. 2009; 34:1487-9.
33. Coquand-Gandit M, Jacob M P, Fhayli W, et al. Chronic Treatment with Minoxidil Induces Elastic Fiber Neosynthesis and Functional Improvement in the Aorta of Aged Mice. Rejuvenation Res. 2017; 20:218-30.
34. Slove S, Lannoy M, Behmoaras J, et al. Potassium channel openers increase aortic elastic fiber formation and reverse the genetically determined elastin deficit in the BN rat. Hypertension. 2013; 62:794-801.
35. Tajima S, Hayashi A, Suzuki T, et al. Stimulation of elastin expression by minoxidil in chick skin fibroblasts. Arch Dermatol Res. 1995; 287:494-7.
36. Hayashi A, Suzuki T, Wachi H, et al. Minoxidil stimulates elastin expression in aortic smooth muscle cells. Arch Biochem Biophys. 1994; 315:137-41.
37. Raveaud S, Mezin P, Lavanchy N, et al. Effects of chronic treatment with a low dose of nicorandil on the function of the rat aorta during ageing. Clin Exp Pharmacol Physiol. 2009; 36:988-94.
38. Li D D, Zhang W, Wang Z Y, et al. Serum Copper, Zinc, and Iron Levels in Patients with Alzheimer's Disease: A Meta-Analysis of Case-Control Studies. Front Aging Neurosci. 2017; 9:300.
39. Harris E D, Rayton J K, Balthrop J E, et al. Copper and the synthesis of elastin and collagen. Ciba Found Symp. 1980; 79:163-82.

While the invention has been described in conjunction with the illustrated embodiments, it will be understood that they are not intended to limit to those embodiments. On the contrary, the invention is intended to cover all alternatives, modifications, and equivalents, which may be included within the invention as defined by the appended claims.

1. A composition for use in a method for the treatment of lung emphysema and other forms of COPD comprising an active agent comprising a copper compound, and a glycosaminoglycan or a physiologically acceptable salt thereof.

2. The composition according to of claim 1, wherein the treatment of lung emphysema and other forms of COPD comprises treatment of a condition of airway wall thickening, bronchiectasis, chronic bronchitis and/or small airways disease.

3. The composition of claim 1, wherein the composition is used for the treatment of a mammal, in particular an adult human being.

4. The composition of claim 1, wherein the active agent comprising a copper compound is a physiologically acceptable copper salt which includes copper sulfate, copper chloride, copper gluconate, copper acetate, copper heptanoate, copper oxide, copper methionate, dicopper oxide, copper chlorophyllin, and calcium copper edetate.

5. The composition of claim 1, wherein the glycosaminoglycan or salt has an average molecular weight of from 12 to 18 kilodaltons.

6. The composition of claim 1, wherein the glycosaminoglycan is heparin.

7. The composition of claim 1, wherein the sodium salt of heparin or heparin sulfate is used.

8. The composition according to any one of the previous claims of claim 1, wherein the dosage of copper component is between 1 μg and 10 mg per day, preferably between 50 μg and 2 mg per day, more preferably between 100 μg and 1 mg per day and most preferably between 200 and 500 μg per day, and the dosage of the glycosaminoglycan component is between 100 and 1,500,000 IU per day, preferably between 5,000 and 1,000,000 IU per day, more preferably between 25,000 and 500,000 IU per day and most preferably between 50,000 and 250,000 IU per day.

9. The composition of claim 1, wherein the doses of the copper component and the glycosaminoglycan component are administered once, twice or three times a day, preferably once a day.

10. The composition of claim 1, wherein the therapeutically active components which constitute the composition are administered simultaneously.

11. The composition of claim 1, wherein the therapeutically active components which constitute the composition are administered sequentially or separately.

12. The composition of claim 1, further comprising at least one medicament or substance with effect on elastin metabolism in the vasculature, selected from the polyphenols epigallocatechin-(3-)gallate (EGCG) and pentagalloyl glucose (PGG), ATP-dependent potassium-channel openers, e.g. minoxidil, nicorandil, diazoxide, pinacidil, and cromakalin, magnesium, vitamin K1, vitamin K2, breakers of AGEs in arteries, e.g. amino-guanidine, pyridoxamine, N-phenacylthiazolium bromide, alagebrium, and flavonoids (e.g. kaempferol, genistein, quercitrin, quercetin, and epicatechin), and/or at least one compound with potential effect on elastin metabolism in the lungs, selected from vitamin A, vitamin D and penta galloyl glucose.

13. The composition of claim 1, wherein the composition is administered via inhalation and/or via instillation.

14. The composition of claim 1, wherein the composition is:
(a) for the reactivation of pulmonary elastin fiber production in a subject;
(b) repair of damaged elastin fibers;
(c) deceleration of the rate of elastin degradation; and/or
(d) inhibition of advanced glycation end product (AGE).

15. The composition of claim 1, wherein the composition is used as an additive to standard pharmacological COPD treatment which includes bronchodilators and immune-modulators, including inhaled corticosteroids and oral macrolides.

16. A method of treatment of a subject suffering from lung emphysema or another form of COPD which comprises administering to said subject a therapeutically active amount of a composition an active agent comprising a copper compound, and a glycosaminoglycan or a physiologically acceptable salt thereof.
